(19) United States
(12) Patent Application Publication
(10) Pub. No.: US 2019/0209519 A1
(43) Pub. Date: Jul. 11, 2019

(54) TOCOTRIENOL COMPOSITION

(71) Applicant: Zenvei International, Inc., Layton, UT (US)

(72) Inventors: Morgan Coleman, Provo, UT (US); Jay Sampson, Uintah, UT (US)

(73) Assignee: Zenvei International, Inc., Layton, UT (US)

(21) Appl. No.: 16/246,393

(22) Filed: Jan. 11, 2019

Related U.S. Application Data
(60) Provisional application No. 62/616,408, filed on Jan. 11, 2018.

(51) Int. Cl. A61K 31/355; A23G 1/42; A23G 1/40; A23G 1/00

(52) U.S. Cl. CPC A61K 31/355; A23G 1/426; A23G 1/40; A23G 1/0036; A23G 1/002; A23V 2002/00; A23V 2300/31; A23V 2200/308; A23V 2200/322; A23V 2200/3262; A23V 2250/60; A23V 2250/712; A23V 2300/24; A23V 2200/02

(57) ABSTRACT

Some embodiments include a chocolate and tocotrienol composition. In some embodiments, a chocolate and tocotrienol composition may be prepared by a process comprising: mixing melted cocoa butter with about 200 mg of tocotrienol at a temperature of about 57° C.; mixing the mixed melted cocoa butter and tocotrienol mixture with a cocoa mass to create a chocolate and tocotrienol composition; and conching the chocolate and tocotrienol composition. Various other embodiments are also disclosed.

### FIELD

This disclosure relates generally to chocolate and tocotrienol compositions.

### BACKGROUND

Vitamin E is a collective name for a family of eight related fat-soluble compounds commonly found in vegetation and seeds with distinctive antioxidant activities, namely the four isomers of tocopherols and four isomers of tocotrienols. The tocotrienol subfamilies are each composed of alpha, beta, gamma and delta that have varying levels of biological activity. Research studies indicate that antioxidants protect cells from the damaging effects of free radicals, which are molecules containing an unpaired electron, and are known to contribute to the development of cancer and cardiovascular disease.

Vitamin E has emerged as an essential fat-soluble nutrient that functions as an antioxidant in the human body to prevent disease and to promote health. Previously, the alpha-tocopherol has been considered to be the most active form. However, recent years of scientific research show that tocotrienols are different from the commonly used vitamin E form i.e. alpha-tocopherol and confer properties that are stronger and often unique from tocopherols. Tocotrienols are distributed throughout the human body via the bloodstream, with particular accumulations found in various body tissues including the brain, heart, skin, cardiac muscle, liver, and adipose tissue after oral dosing. Therefore, recent developments suggest that the tocotrienol subfamily of natural vitamin E is better antioxidant than tocopherol subfamily as having powerful neuroprotective, tumor suppressive effect and cholesterol lowering properties.

Dark chocolate has been shown to have a number of health benefits. These benefits may include high levels of fiber and/or minerals. In addition, dark chocolate has been found to be a powerful source of antioxidants. Dark chocolate also has been found to improve blood flow and/or lower blood pressure. Lastly, dark chocolate has been found to raise HDL and protects LDL against oxidation, lower risk of cardiovascular disease, and/or protect your skin against the sun.

### SUMMARY

Some embodiments include a chocolate and tocotrienol composition. In some embodiments, a chocolate and tocotrienol composition may be prepared by a process comprising: mixing melted cocoa butter with about 200 mg of tocotrienol at a temperature of about 57° C.; mixing the mixed melted cocoa butter and tocotrienol mixture with a cocoa mass to create a chocolate and tocotrienol composition; and conching the chocolate and tocotrienol composition. Various other embodiments are also disclosed.

### BRIEF DESCRIPTION OF THE FIGURES

These and other features, aspects, and advantages of the present disclosure are better understood when the following Detailed Description is read with reference to the accompanying drawings.

FIG. 1 is a flowchart of a process for creating a chocolate and tocotrienol composition according to some embodiments.

### DETAILED DESCRIPTION

Chocolate and tocotrienol compositions and method for producing chocolate and tocotrienol compositions are disclosed. In some embodiments, tocotrienol can be combined with chocolate to produce a dietary supplement that improves health and has a high ingestion rate.

One of the possible challenges associated with a chocolate and tocotrienol composition is taste. Tocotrienol does not have a pleasing flavor whereas chocolate does. The challenge is to manufacture a chocolate and tocotrienol composition that has the flavor of chocolate rather than tocotrienol. Another possible challenge is related to producing a chocolate and tocotrienol composition that has a satisfactory consistency.

In some embodiments, a chocolate and tocotrienol composition can include tocotrienol in a chocolate composition. For example, a dosage of tocotrienol can be included, mixed, emulsified, combined, embedded, dissolved, blended, etc. within melted chocolate prior to solidification.

In some embodiments, the amount of tocotrienol within a chocolate and tocotrienol composition can include 5 mg, 10 mg, 25 mg, 50 mg, 100 mg, 150 mg, 200 mg, 250 mg, 500 mg, etc. of tocotrienol. The amount of tocotrienol can be used with 1 g, 2 g, 2.5 g, 5 g, 10 g, etc. chocolate. In some embodiments, the chocolate may comprise a high cocoa chocolate such as, for example, chocolate with more than 60%, 65%, 70%, 75%, 80%, 85%, 90%, etc. cocoa content.

In some embodiments, the tocotrienol can include alpha tocotrienol, beta tocotrienol, gamma tocotrienol, or delta tocotrienol. In some embodiments, the tocotrienol may be derived from palm oil, rice bran oil, wheat germ, barley, saw palmetto, anatto, or any other type of seed, nut, grain, and/or the oils derived therefrom, etc.

In some embodiments, a given type of tocotrienol isomer may demonstrate different bioavailability and/or efficacy than another tocotrienol isomer depending on the type of antioxidant performance being measured. In some embodiments, a tocotrienol isomer may have some physical antioxidant activity due to an ability to donate a hydrogen atom (a proton plus electron) from the hydroxyl group on the chromanol ring, to free radical and reactive oxygen species.

In some embodiments, the chocolate and tocotrienol composition can be manufactured in a number of ways. For example, a chocolate liquor can be combined with sugar, cocoa butter, flavor (e.g., vanilla), milk, and/or other ingredients (e.g., tocotrienol oil, powder, or granules). This chocolate mixture may be run through a series of steel rollers, for example, to refine the texture.

The chocolate mixture may then be conched. The mixture may be conched, for example, by mixing, mashing, swirling, and aerating the chocolate mixture. While the mixture is being conched, the mixture may also be combined with cocoa butter, soy lecithin, or other ingredients (e.g., tocotrienol oil, powder, or granules), etc. In some embodiments, the chocolate mixture may be conched for a few hours, or a few days.

After conching, the chocolate may be tempered by stirring it, cooling it, and heating it back up slowly. The chocolate mixture may be tempered a plurality of times. In some embodiments, tocotrienol oil, powder, or granules may be added during one or more of the conching steps.

The chocolate may then be formed into a shape such as, for example, a bar, square, sphere, roll, dollop, etc. and cooled to a hardened form. Lastly, the hardened chocolate may be wrapped and/or packaged.

Tocotrienol oil, tocotrienol powder, or tocotrienol granules may be added to the chocolate mixture during or before either or both the conching and the tempering steps.

For example, tocotrienol oil, tocotrienol powder, or tocotrienol granules may be added to the chocolate mixture at any time during the manufacturing process.

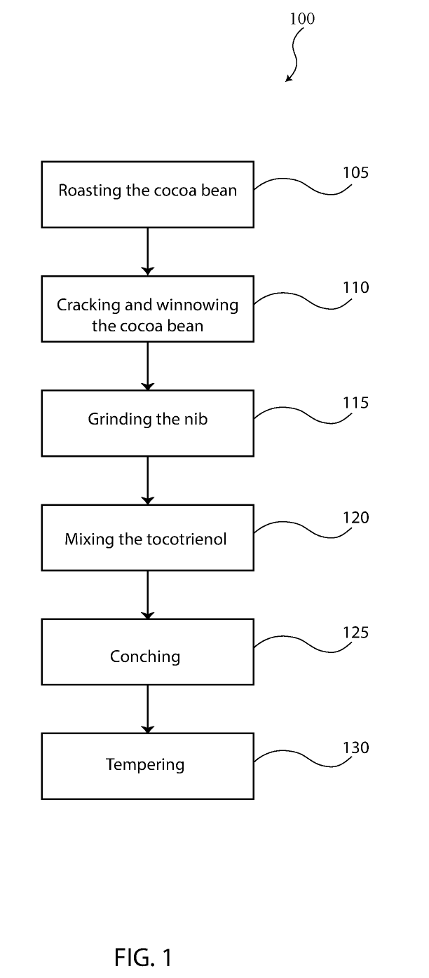

FIG. 1 is a flowchart of a process 100 for creating a chocolate and tocotrienol composition according to some embodiments.

Process 100 starts at block 105. At block 105 cocoa beans may be roasted. In some embodiments, the cocoa bean may be roasted to bring out the characteristic chocolate aroma. In some embodiments, the beans may be roasted in large rotary cylinders. In some embodiments, the roasting may last for any period of time such as, for example, from 30 minutes to two hours. In some embodiments, the cocoa may be roasted at temperatures of 250 degrees Fahrenheit or higher. In some embodiments, during roasting, the beans may be turned over and over, the moisture content within the bean drops, the color changes to a rich brown, and/or the characteristic aroma of chocolate may become evident.

At block 110 the cocoa beans may be cracked and/or winnowed. In some embodiments, after roasting the cocoa beans may be cooled and the thin shells, which have become brittle by roasting, may be removed. In some embodiments, a winnowing machine may pass the beans between serrated cones so the cocoa beans are cracked rather than crushed. For example, a series of mechanical sieves may separate the broken pieces into large and small grains while fans blow away the thin, light shell from the remaining pieces (or “nibs”). In some embodiments, nibs from different varieties of cocoa beans may be combined.

At block 115 the nibs may be ground. In some embodiments, the nibs may pass through refining mills and are ground between large grinding stones or heavy steel discs creating a cocoa mass. In some embodiments, the cocoa mass may be subjected to hydraulic pressure causing cocoa butter to flow out from the cocoa mass.

In some embodiments, the cocoa mass may be further refined and/or processed.

At block 120 tocotrienol and/or cocoa butter may be mixed with the cocoa mass creating a chocolate and tocotrienol mixture. In some embodiments, melted cocoa butter may be used. In some embodiments, the cocoa butter may be melted and mixed with tocotrienol oil prior to mixing with the cocoa mass. In some embodiments, other ingredients may also be mixed with the cocoa mass such as, for example, vanilla, sugar, allulose, flavors, milk, sweeteners, etc.

In some embodiments, the tocotrienol and/or cocoa butter may be mixed with the cocoa mass at a temperature between 50° C.-65° C. such as, for example, 57° C.

In some embodiments, the tocotrienol and/or cocoa butter may be mixed with the cocoa mass within a mixer such as, for example, with rotating and/or kneading arms.

In some embodiments, the tocotrienol and/or cocoa butter may be mixed with the cocoa mass within a refining machine comprising rollers.

In some embodiments, the tocotrienol and/or cocoa butter may be mixed with the cocoa mass within a conching machine.

At block 125 the chocolate and tocotrienol mixture can be conched. In some embodiments, conches may be equipped with heavy rollers that plow back and forth through the chocolate mixture, anywhere from a few hours to several days. In some embodiments, the conching can grind the chocolate particles extremely fine (e.g., down below 20 microns), which can reduce conching times. In some embodiments, the chocolate mixture may be conched for 4 to 12 hours or up to 96 hours.

Any type of conching process and/or conching machine may be used.

At block 130 the chocolate and tocotrienol mixture may be tempered. In some embodiments, tempering may alternately heat and/or cool the chocolate and tocotrienol mixture.

In some embodiments, after tempering the chocolate and tocotrienol mixture may be formed into shapes.

Any reference within this disclosure of a tocotrienol can be replaced with a tocopherol.

The term “substantially” or the term “about” means within 5% or 10% of the value referred to or within manufacturing tolerances.

Various embodiments are disclosed. The various embodiments may be partially or completely combined to produce other embodiments.

Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for-purposes of example rather than limitation, and does not preclude inclusion of such modifications, variations, and/or additions to the present subject matter as would be readily apparent to one of ordinary skill in the art.

That which is claimed:

1. A method comprising:
grinding cocoa nibs;
separating cocoa butter from the cocoa nibs to produce a cocoa mass;
mixing the cocoa butter with tocotrienol;
mixing the mixed cocoa butter and tocotrienol with the cocoa mass to create a chocolate and tocotrienol composition; and
conching the chocolate and tocotrienol composition.

2. The method according to claim 1, wherein the cocoa butter is heated to a temperature of about 57° C.

3. The method according to claim 1, wherein the tocotrienol comprises tocotrienol oil.

4. The method according to claim 1, further comprising melting coca butter prior to mixing the cocoa butter with tocotrienol.

5. The method according to claim 1, wherein the cocoa mass comprises cocoa liquor.

6. The method according to claim 1, further comprising mixing allulose with the cocoa butter and the tocotrienol.

7. The method according to claim 1, wherein the conching comprises dry conching.

8. The method according to claim 1, wherein the tocotrienol comprises 2.3% of the total mass of the total chocolate and tocotrienol composition.

9. The method according to claim 1, wherein the mass of the tocotrienol comprise about 200 mg and the mass of the chocolate and tocotrienol composition comprises about 8 grams to about 9 grams.

10. The method according to claim 1, wherein mixing the cocoa butter with tocotrienol further comprises mixing additional cocoa butter with the cocoa butter and the tocotrienol.

11. A chocolate and tocotrienol composition prepared by a process comprising:
mixing melted cocoa butter with about 200 mg of tocotrienol at a temperature of about 57° C.;
mixing the mixed melted cocoa butter and tocotrienol mixture with a cocoa mass to create a chocolate and tocotrienol composition; and
conching the chocolate and tocotrienol composition.

12. The chocolate and tocotrienol composition according to claim 11, wherein the tocotrienol comprises tocotrienol oil.

13. The chocolate and tocotrienol composition according to claim 11, wherein the cocoa mass comprises cocoa liquor.

14. The chocolate and tocotrienol composition according to claim 11, further comprising mixing allulose with the cocoa butter and the tocotrienol.

15. The chocolate and tocotrienol composition according to claim 11, wherein the conching comprises dry conching.

16. The chocolate and tocotrienol composition according to claim 11, wherein the tocotrienol comprises 2.3% of the total mass of the total chocolate and tocotrienol composition.

17. The chocolate and tocotrienol composition according to claim 11, wherein the mass of the tocotrienol comprise about 200 mg and the mass of the chocolate and tocotrienol composition comprises about 8 grams to about 9 grams.

18. A chocolate and tocotrienol composition prepared by a process comprising:
grinding cocoa nibs;
separating cocoa butter from the cocoa nibs to produce a cocoa mass;
melting the cocoa butter;
mixing the melted cocoa butter with about 200 mg of tocotrienol oil at a temperature of about 57° C.;
mixing the mixed melted cocoa butter and tocotrienol oil mixture with the cocoa mass to create a chocolate and tocotrienol composition; and
dry conching the chocolate and tocotrienol composition,
wherein the chocolate and tocotrienol composition comprises a mass of about 8 grams to about 9 grams.
